(19) United States
(12) Patent Application Publication
(10) Pub. No.: US 2021/0378483 A1
(43) Pub. Date: Dec. 9, 2021

(54) CONTROL AND INPUT DEVICE FOR MEDICAL OBSERVATION DEVICE INCORPORATING POINTING DEVICE WITH CO-LOCATED SOFT AND HARD INPUTS AND GRAPHICAL USER INTERFACE

(71) Applicant: OLYMPUS CORPORATION, Tokyo (JP)

(72) Inventor: Takehiro YOSHIMURA, Tokyo (JP)

(73) Assignee: OLYMPUS CORPORATION, Tokyo (JP)

(21) Appl. No.: 17/336,920

(22) Filed: Jun. 2, 2021

Related U.S. Application Data
(60) Provisional application No. 63/034,054, filed on Jun. 3, 2020.

(51) Int. Cl. A61B 1/00; G06F 3/033; G06F 3/0481; G06F 3/0484; G06F 3/01

(52) U.S. Cl. CPC A61B 1/00006; G06F 3/033; A61B 1/00188; G06F 3/04842; G06F 3/016; G06F 3/04812

(57) ABSTRACT

An input device for operating a medical observation system having, arranged on an interface surface, a pointing device, hardware buttons in spaced apart areas, and a touch display panel including a graphical user interface with interchangeable software buttons. The hardware buttons and software buttons have specific functions, such as control of the pointing device, and are placed surrounding and adjacent to the periphery of the pointing device. This configuration helps the operator to simultaneously and singlehandedly operate the input device and without devoting substantial focus toward the input device itself. Various features to allow the operator to distinguish the hardware and software buttons having specific functions are presented, including tactile and haptic features, illumination effects, and other visual effects.

### RELATED APPLICATION DATA

This application is based on and claims priority under 35 U.S.C. § 119 to U.S. Provisional Application No. 63/034,054, filed Jun. 3, 2020, the entire contents of which are incorporated herein by reference.

### FIELD OF DISCLOSURE

The present disclosure relates to an input device for a medical observation device having a pointing device, hardware buttons, and a touch panel including a graphical user interface with interchangeable software buttons. In order to medically observe or inspect a patient, an optics or ultrasound endoscope including an optical camera or ultrasound transducer placed at a distal end of an insertion portion may be used to generate an optic or ultrasound image. In a medical procedure or diagnosis using an optics or ultrasound endoscope, an image generated by the medical observation device based on the optical image or ultrasound echo may be displayed on a display device. Settings of an observation mode and an observation condition during the procedure, as well as the operation related to the optical or ultrasound image, are performed based on the instruction signal created using the input device. The present disclosure also relates to a method of operating such an input device for a medical observation device.

### BACKGROUND

In the discussion that follows, reference is made to certain structures and/or methods. However, the following references should not be construed as an admission that these structures and/or methods constitute prior art. Applicant expressly reserves the right to demonstrate that such structures and/or methods do not qualify as prior art against the present invention.

A medical practitioner generally operates an input device for a medical observation device, such as an endoscope, during a medical procedure, such as the diagnosis of a patient. During the diagnosis procedure, the operator would mainly view the display connected to the medical observation system or the diagnosed patient, with one hand operating the medical equipment, such as an optical endoscope or an ultrasound endoscope. With the one hand occupied with manipulation of the medical equipment and a need to focus on the display or the patient being diagnosed rather than on the input device, an efficient input device enabling simple and easy operation of the medical observation system is desired.

FIG. 13 is a figure of an input device used in an ultrasonic observation device in the related art (Japanese Patent Laid-open Publication No. 2007-202652). The related art input device 10 includes trackball 14 and trackball related operating buttons 15 configured to operate the ultrasonic observation device. The related art input device 10 is configured to allow the medical practitioner to operate the ultrasonic observation device using a single hand, by placing the trackball related operation buttons 15 within the reachable peripheral area adjacent to the trackball 14. A drawback of the related art input device 10 is that the trackball related operating buttons 15 are all hardware buttons in which each hardware button is assigned a single function. This configuration of the related art input device 10 limits the number of trackball related operating buttons 15 placed adjacent to the trackball 14, which is problematic since the number of functions related to the trackball operation cannot increase or change as the capabilities of the medical observation system increases or changes. Other features shown in FIG. 13 include keyboard 11, slide controls 12 and input buttons 13.

FIG. 14 is a figure of another input device used in an ultrasonic observation device in the related art (United States Patent Application Publication No. US2019/02544631A1). The related art input device 50 includes touch pad 51 and display unit 52. The display unit 52 of the related art may constitute a touch panel that can display buttons having assignments related to the function of the touch pad 51. The drawback of the related art input device is that buttons displayed on the display unit 52 are not assigned a particular region or are not distinguishable to the other displayed buttons. In addition, operation of the related art input device 50 by one finger placed on the touch pad 51 (see movement of hand (h)), while at the same time operating the buttons controlling the function of the touch pad 51 to instantly see the effect of the changes made on the display, is limited in input device 50. In addition, it would be an improvement for the buttons controlling touch pad 51 to be easily distinguishable compared to the other unrelated displayed buttons. Thus, the display region of the buttons and how to distinguish the touch pad related buttons would improve the usability of the related art input device 50.

### SUMMARY

Accordingly, there is a need for an input device with improved operability and that can substantially obviate one or more of the issues due to limitations and disadvantages of related art input devices.

An object of the present disclosure is to provide an improved input device that provides an efficient input system with simple and easy to use configuration for medical practitioners compared to the related art. At least one or some of the objectives is achieved by the input device disclosed herein.

Additional features and advantages will be set forth in the description that follows, and in part will be apparent from the description, or may be learned by practice of the invention. The objectives and other advantages of the disclosed input device will be realized and attained by the structure particularly pointed out in the written description and claims thereof, as well as the appended drawings.

Embodiments of the disclosed input device comprises an interface surface including a pointing device configured to input an operation to a medical observation device. The disclosed interface surface includes a first area that is located adjacent a first portion of a periphery of the pointing device and that includes a first plurality of hardware buttons configured to control functions of the medical observation device. The interface surface also includes a second area that is located adjacent a second portion of the periphery of the pointing device and that also includes a second plurality of hardware buttons configured to control functions of the medical observation device. The interface surface also includes a touch panel including a graphical user interface having an input display region containing at least one software button configured to control a function of the medical observation device. The input display region is located at a first edge portion of the graphical user interface and the first edge portion of the graphical user interface is located adjacent a third portion of the periphery of the pointing device. Said third portion of the periphery is interposed between the first portion of the periphery and the second portion of the periphery of the pointing device.

According to one embodiment, a fourth portion of the periphery is interposed between the first portion of the periphery and the second portion of the periphery, along the periphery of the pointing device.

According to one embodiment, an area adjacent the fourth portion of the periphery is free of hardware buttons configured to control functions of the medical observation device and is free of software buttons configured to control functions of the pointing device.

According to one embodiment, the first portion of the periphery and the second portion of the periphery are at opposing sides of the pointing device.

In some embodiments, the fourth portion is interposed between the first portion of the periphery and the second portion of the periphery in a first direction around the periphery of the pointing device and the third portion of the periphery is interposed between the first portion of the periphery and the second portion of the periphery in a second direction around the periphery of the pointing device. In other embodiments, in one direction around the periphery of the pointing device, the third portion and the fourth portion are both interposed between the first portion of the periphery and the second portion of the periphery.

According to one embodiment, a number of hardware buttons in the first area is different from a number of hardware buttons in the second area.

According to one embodiment, at least one of the first plurality of hardware buttons is configured to control the operation inputted to the medical observation device.

According to one embodiment, at least one of the second plurality of hardware buttons is configured to control the operation inputted to the medical observation device.

According to one embodiment, the operation of the pointing device inputted by the at least one hardware button is used in a plurality of modes of the medical observation device.

According to one embodiment, the operation of the pointing device inputted by the hardware buttons are selected from the group consisting of a pointer function, a measurement function, a set function, a comment function, and a delete function.

According to one embodiment, the function of the pointing device associated with at least one software button changes in accordance with the mode of the medical observation device.

According to one embodiment, the mode of the medical observation device is selected using a plurality of mode buttons arranged in a mode display region of the graphical user interface.

According to one embodiment, the function of the pointing device associated with the at least one software button is selected from the group consisting of scroll, rotation, focus change depth, ROI position change, and ROI size change.

According to one embodiment, the function of the pointing device associated with the at least one software button changes based on a type of the medical observation device connected to the input device.

According to one embodiment, the input display region of the graphical user interface is located at a twelve o'clock position relative to the periphery of the pointing device.

According to one embodiment, the first area is located at a nine o'clock position relative to the periphery of the pointing device and the second area is located at a three o'clock position relative to the periphery of the pointing device.

According to one embodiment, the interface surface associated with one or more of (i) the pointing device, (ii) at least one of the first plurality of hardware buttons, (iii) at least one of the second plurality of hardware buttons, and (iv) the at least one software button includes a tactile feature.

According to one embodiment, the interface surface associated with one or more of (i) the pointing device, (ii) at least one of the first plurality of hardware buttons, (iii) at least one of the second plurality of hardware buttons, and (iv) the at least one software button includes a haptic feedback feature.

According to one embodiment, the pointing device is one of a touch pad, a track ball, a pointing stick, or a joystick.

According to one embodiment, the hardware and the software buttons controlling the function of the pointing device has the same shape.

According to one embodiment, the first area of the interface surface, the second area of the interface surface, and the input display region of the graphical user interface are all at a position on the interface surface that is within 15 cm from a center of the pointing device.

According to one embodiment, the first area of the interface surface, the second area of the interface surface, and the input display region of the graphical user interface are all co-located within an operating area of the interface surface operable by a hand of an operator without changing a location of the hand relative to the interface surface.

According to one embodiment, the first area of the interface surface, the second area of the interface surface, and the input display region of the graphical user interface are all co-located within an operating area of the interface surface of less than 100 cm².

According to one embodiment, the operating area of the interface surface is less than 90 cm².

According to one embodiment, a shape of the operating area of the interface surface is selected from one of a quadrilateral, a circle and an oval.

According to one embodiment, a medical observation system comprises a medical device, such as an endoscope, a medical observation device connected to the medical device, and any one of the embodiments of the input device disclosed herein.

In exemplary embodiments, the disclosed input device can be an utilized in a method of medical treatment or method of medical diagnosis, in which method an operator (i) inputs an operation to a medical observation device via a pointing device arranged on the interface surface of the input device, (ii) controls functions of the medical observation device via a first plurality of hardware buttons arranged in a first area of the interface surface located adjacent a first periphery of the pointing device, (iii) controls functions of the medical observation device via a second plurality of hardware buttons arranged in a second area of the interface surface located adjacent a second periphery of the pointing device, and (iv) controls functions of the medical observation device via at least one software button contained in an input display region of a graphical user interface included in a touch panel arranged on the interface surface of the input device, where the input display region is located at a first edge portion of the graphical user interface and the first edge portion of the graphical user interface is located adjacent a third portion of the periphery of the pointing device, and where the third portion of the periphery is interposed between the first portion of the periphery and the second portion of the periphery of the pointing device.

Other systems, methods, features and advantages will be, or will become, apparent to one with skill in the art upon examination of the following figures and detailed description. It is intended that all such additional systems, methods, features and advantages be included within this description, be within the scope of the present disclosure, and be protected by the following claims. Nothing in this section should be taken as a limitation on those claims. Further aspects and advantages are discussed below in conjunction with the embodiments of the disclosed input device. It is to be understood that both the foregoing general description and the following detailed description of the disclosed input device are examples and explanatory, and are intended to provide further explanation of the disclosed input device as claimed.

### BRIEF DESCRIPTION OF THE DRAWING

The following detailed description of preferred embodiments can be read in connection with the accompanying drawings in which like numerals designate like elements and in which:

FIG. 1 is a block diagram illustrating a configuration of a medical observation system that includes an ultrasonic observation device according to an embodiment of the present disclosure.

FIG. 2 illustrates a form of usage of the medical observation system illustrated in FIG. 1.

FIG. 3 illustrates input device illustrating touch pad, touch panel, and hardware buttons.

FIG. 4 illustrates input device illustrating various display regions and software buttons.

FIG. 5 illustrates input device with software buttons in a different shape.

FIGS. 6 and 7 illustrates input device with different set of software buttons according to the observation mode.

FIGS. 8 and 9 illustrate input device with different set of software buttons according to the connected medical equipment.

FIG. 10 illustrate input device with active buttons and inactive buttons.

FIGS. 11 and 12 illustrate flowcharts describing an operation of the active and inactive buttons.

FIGS. 13 and 14 are figures from the discussed prior art.

For ease of viewing, in some instances only some of the named features in the figures are labeled with reference numerals.

### DETAILED DESCRIPTION

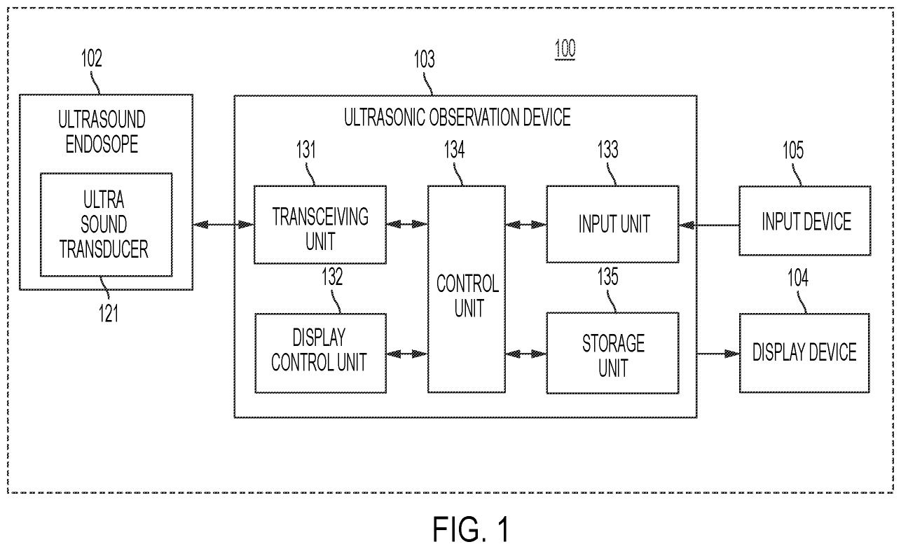

FIG. 1 is a block diagram illustrating a configuration of a medical observation system that includes a medical observation device (in this example, an ultrasonic observation device) according to an embodiment of the present disclosure. As illustrated in FIG. 1, a medical observation system 100 includes an ultrasound endoscope 102 that transmits ultrasonic waves to a subject to be observed, and that receives ultrasonic waves reflected by the subject, an ultrasonic observation device 103 that generates an ultrasound image based on an ultrasonic signal acquired by the ultrasound endoscope 102, a display device 104 that displays the ultrasound image generated by the ultrasonic observation device 103, and an input device 105 that accepts input of an instruction signal for observation mode setting, observation condition setting, or the like with respect to the medical observation device.

The ultrasound endoscope 102 has, at its distal end portion, an ultrasound transducer 121. The ultrasound transducer 121 converts an electrical pulse signal received from the ultrasonic observation device 103 into ultrasonic pulses (acoustic pulses) and irradiates the ultrasonic pulses to the subject. Then, the ultrasound transducer 121 receives ultrasonic echoes reflected by the subject, converts the received ultrasonic echoes into an electrical echo signal (ultrasonic wave signal) in which an ultrasonic echo is expressed by, e.g., voltage variations, and outputs the electrical echo signal to the ultrasonic observation device 103.

The ultrasonic observation device 103 includes a transceiving unit 131, a display control unit 132, an input unit 133, a control unit 134, and a storage unit 135.

The transceiving unit 131 performs transmission and reception of an electrical signal with the imaging device and the ultrasound transducer 121. The transceiving unit 131 is electrically connected to the imaging device, transmits imaging information about imaging timing and the like to the imaging device, and receives an imaging signal generated by the imaging device. Moreover, the transceiving unit 131 is electrically connected to the ultrasound transducer 121, transmits an electrical pulse signal to the ultrasound transducer 121, and receives an echo signal that is an electrical reception signal from the ultrasound transducer 121. Specifically, the transceiving unit 131 generates an electrical pulse signal based on a predetermined waveform and transmission timing, and transmits the generated pulse signal to the ultrasound transducer 121.

The transceiving unit 131 also performs sensitivity time control (STC) correction in which an echo signal having a larger reception depth is amplified at a higher amplification factor. The transceiving unit 131 subjects the amplified echo signal to filtering and the like, and then generates a digital high frequency (radio frequency (RF)) signal of time domain to output.

The display control unit 132 generates endoscopic image data based on an imaging signal and ultrasound image data that corresponds to an electrical echo signal. Furthermore, the display control unit 132 superimposes various kinds of information on the endoscopic image data and the ultrasound image data to output, and controls display of the display device 104. The display control unit 132 uses a central processing unit (CPU) having arithmetic and control functions, various kinds of arithmetic circuits, or the like.

The input unit 133 receives an instruction signal input by the input device 105, and accepts input of various kinds of information according to the received instruction signal. The various kinds of information include observation mode setting information, observation condition setting information (for example, a gain and switching of a display range, scroll specification information (for example, a sliding direction and a sliding amount of a B-mode image)), rotation specification information (for example, a rotation direction and a rotation amount of an ultrasound image), and the like.

The control unit 134 controls the entire ultrasonic diagnostic system 100. The control unit 134 uses a CPU having arithmetic and control functions, various kinds of arithmetic circuits, and the like. The control unit 134 reads information stored in the storage unit 135, and overall-controls the ultrasonic observation device 103 by performing various kinds of arithmetic processing related to an operating method of the ultrasonic observation device 103. Note that the control unit 134 may also be realized by the CPU or the like that constitutes the display control unit 132 (and vice versa).

The storage unit 135 stores various kinds of programs to operate the medical observation system 100, data including various kinds of parameters necessary for operation of the medical observation system 100, and the like. The storage unit 135 stores, for example, an initial position (sound ray number) of a write start position (transmission start position of ultrasonic wave) of an ultrasound image.

Moreover, the storage unit 135 stores various kinds of programs including an operating program to perform an operating method of the ultrasonic diagnostic system 100. It is possible to distribute the operating program by storing the program in a computer-readable recording medium, such as a hard disk, a flash memory, a compact-disk read-only memory (CD-ROM), a digital versatile disk read-only memory (DVD-ROM), and a flexible disk. It is also possible to acquire the various kinds of programs described above by downloading through a communication network. The communication network herein can be realized by, for example, an existing public line network, a local area network (LAN), a wide area network (WAN), or the like, and it does not matter whether the communication network is wired network or wireless network.

The storage unit 135 having the configuration as above can be realized by using a read-only memory (ROM) in which various kinds of programs are installed in advance, a random access memory (RAM) in which arithmetic parameters and data of respective processing are stored, and the like.

The display device 104 is connected to the ultrasonic observation device 103. The display device 104 is configured with a display panel constituted of a liquid crystal display, an organic electroluminescence (EL) display, or the like. In the illustrated embodiment, the display device 104 displays an ultrasound image output by the ultrasonic observation device 103, and various kinds of information relating to operation.

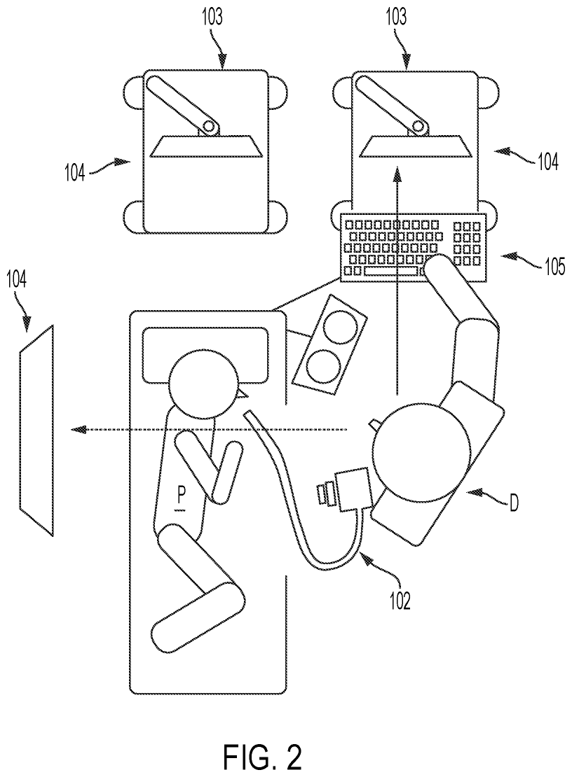

FIG. 2 illustrates an example medical observation device as in FIG. 1 in an in-use arrangement, e.g., as a medical observation device in a diagnostic procedure by a medical professional on a patient. As illustrated in FIG. 2, the operator, in this case a doctor (D), uses one hand (e.g., a left hand) for operating the ultrasound endoscope 102 and the other hand (e.g., a right hand) for operating the input device 105. The operator will mainly focus on the display device 104 and the patient (P) through the diagnostic procedure. Because the operator's attention is required to be focused elsewhere, an input device 5 with an intuitive design that is easy to operate single handedly is beneficial to the operator.

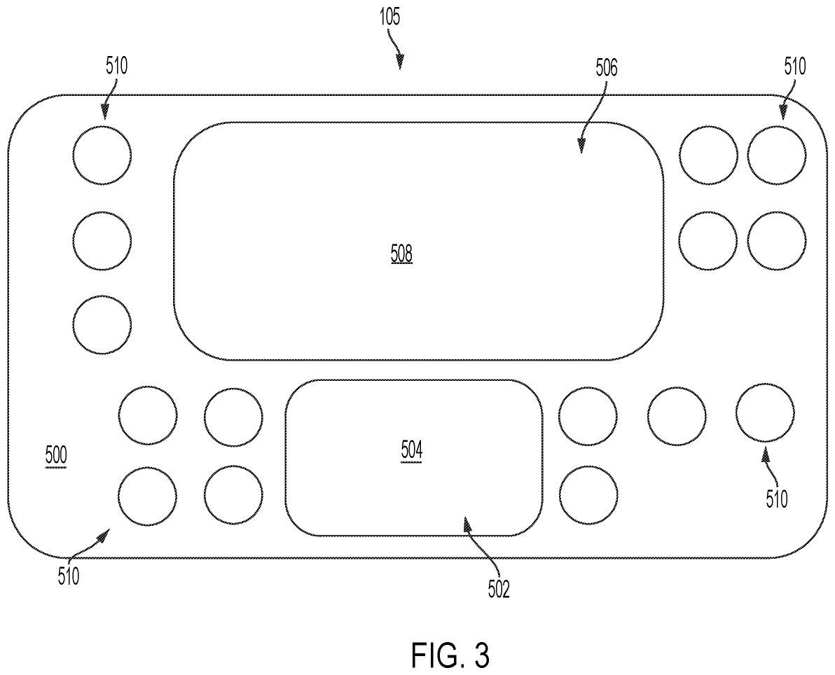

FIG. 3 illustrates a configuration of the input device 105. As illustrated in FIG. 3, the input device 105 includes an interface surface 500. The input device 105 includes, arranged on the interface surface 500, a pointing device 502, such as a touch pad 504 that detects a contact of a finger of an operator or the like, and a touch panel 506. Touch panel 506 is capable of displaying various kinds of information, and serves as a touch panel for inputting instructions from the operator.

The input device 105 also includes, arranged on the interface surface 500, several areas arranged relative to the pointing device and each containing select input devices, whether in the form of buttons, controls, slides, switches, etc. For example, hardware buttons 510 can be included that allow operators to input instructions, change settings or modes, and otherwise configure and control the operation of the medical observation device. In exemplary embodiments, the input device 105 is connected to the ultrasonic observation device 103 through a cable or through wireless connection, and outputs a signal of instruction to the input unit 133 based on input to the input device 105 from the pointing device 502, touch panel 506, and hardware buttons 510.

For example, the input device 105 detects a contact position by a contact sensor when a contact object, such as a finger of an operator, contacts the pointing device 502, and outputs information on the contact position to the ultrasonic observation device 103. Moreover, when the contact object moves while staying in contact with the pointing device 502, the input device 105 detects a moving direction and a moving amount (such as distance), and outputs information on the moving direction and the moving amount to the ultrasonic observation device 103. The ultrasonic observation device 103 performs signal processing in accordance with the received information. Furthermore, the ultrasonic observation device 103 outputs, for example, an image for which a position of the image to be displayed on the display device 104 is translated or rotated based on the received information.

FIG. 3 illustrate an input device 105 with touch pad 504 as the pointing device 502, which detects the moving direction and moving amount of the contact object (pointing information). The detection of pointing information can be achieved through other pointing devices, such as trackball, pointing stick, or joystick.

The touch panel 506 includes a graphical user interface 508 including an input display region that is configurable, such as to include software buttons and keyboards. The graphical user interface 508 may also display other information (i.e. patient information, medical observation device status information, and information on medical equipment being used) in text and other graphical formats. The information displayed on the graphical user interface 508 may be changed by selecting the observation mode setting, the observation condition setting, and the like using the hardware button 510, or other input methods such as pointing device 502 and touch panel 506.

Each hardware button 510 is allocated a function. The allocated function may be described on the hardware button 510 itself, labeled near or adjacent to the hardware button 510, or may have a representative symbol (not shown). Example functions that may be assigned to the hardware button 510 include a measurement function, a set function, a comment function, and a delete function. The hardware buttons 510 may be comprised of a transparent material and a light source such as a LED, so as to provide illumination of the hardware button 510 in multiple or predesignated colors.

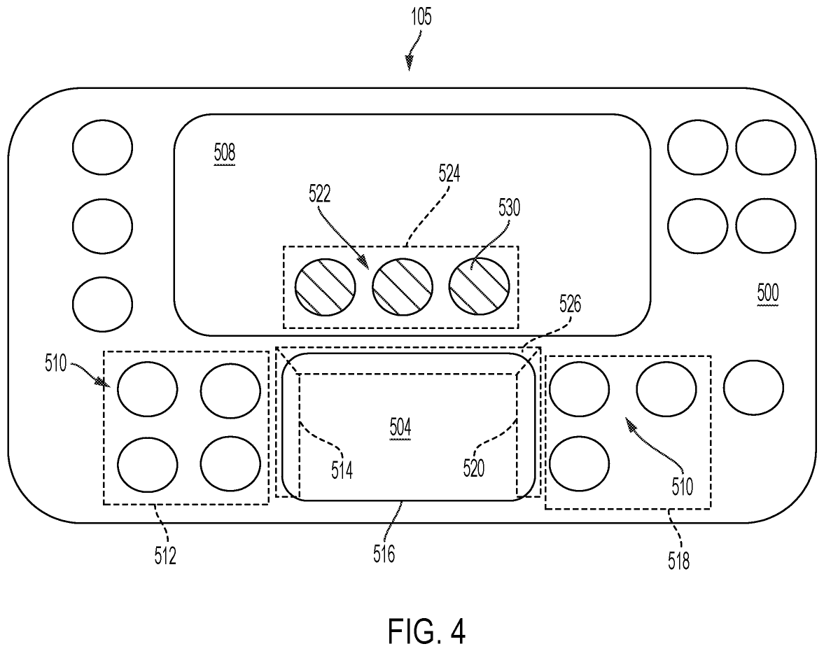

FIG. 4 illustrates an embodiment of a configuration of the input device 105 wherein a set of hardware buttons 510 are grouped together in first area 512, which is directly adjacent to a first portion 514 of the periphery 516 of the pointing device 502 (such as touch pad 504), and a second set of hardware buttons 510 are grouped together in a second area 518, adjacent to a second portion 520 of the periphery 516 of the pointing device 502. The graphical user interface 508 includes an input display region 522 located at a first edge portion 524 of the graphical user interface 508. The first edge portion 524 of the graphical user interface 508 is itself located adjacent to a third portion 526 of the periphery 516 of the pointing device 502.

As readily seen in FIG. 4, along the periphery 516 of the pointing device 502 (such as touch pad 504), the third portion 526 of the periphery 516 is interposed between the first portion 514 of the periphery 516 and the second portion 520 of the periphery 516. In some embodiments where the periphery 516 has a shape of a polygon, such as a parallelogram (such as a quadrilateral), a hexagon, or an octagon, the various portions of the periphery correspond to different sides of the polygon. In other embodiments where the periphery 516 has a shape having a curvature, such as a circle or oval but also an irregular closed curved shape, the various portions of the periphery correspond to different arc lengths of curved shape.

The input display region 522 includes software buttons 530. The hardware buttons 510 in first area 512 and second area 518, as well as software buttons 530 in the input display region 522, may be assigned functions related to operating the medical observation device, e.g., ultrasound endoscope 102, ultrasonic medical observation device 103, display device 104, or the pointing device 502 of the input device 105.

As noted above, the software buttons 530 included in the input display region 522 may be assigned functions related to controlling a function of the medical observation device 103. The functions associated with the control of the medical observation device 103 may include one of scroll, rotation, focus change depth, and region of interest (ROI) parameters, such as ROI position change, or ROI size change. Such control function of the medical observation device 103 may also optionally include controlling the pointing device 502. The software buttons 530 may be interchangeable according to design and observation mode of the medical observation device, allowing the operator to select from almost infinite number of software buttons to be displayed in the input display region 522.

The location of first area 512, second area 518, and input display region 522 may be replaced according to the design of the input device 105 or interface surface 500, granted that the three areas are adjacent to the pointing device 502, preferably within 15 cm from the center of the pointing device. The center of the pointing device is the operational center of the pointing device, which is preferably also the geometric center of the point device. More preferably, the three areas are directly adjacent to the pointing device 502, where directly adjacent means there are no control input devices interposed between the periphery 516 of the pointing device 502 and the respective areas, e.g., first area 512, second area 518, and input display region 522. Co-locating the three areas within an operating area of the interface surface 500 of the input device 105 allows the input devices in the areas, e.g., first area 512, second area 518, and input display region 522, to be operable by a single hand of an operator without changing a location of the hand relative to the input device 105. Said operating area of the interface surface 500 is preferably less than 100 cm² or less than 90 cm². The allocation of the three areas being near the pointing device 502 allows the operator to operate the pointing device 502, hardware buttons 510 in the first area 512, hardware buttons 510 in the second area 518, and software buttons 530 in input display region 522 singlehandedly.

The same configuration may also allow the operator to operate two of more of the pointing device 502, hardware buttons 510 in the first area 512, hardware buttons 510 in the second area 518, and software buttons 530 in input display region 522 simultaneously, for example, by placing one finger on the touch pad 504 and the other fingers on one of a hardware button 510 in the first area 512, a hardware button 510 in the second area 518, and a software button 530 in input display region 522.

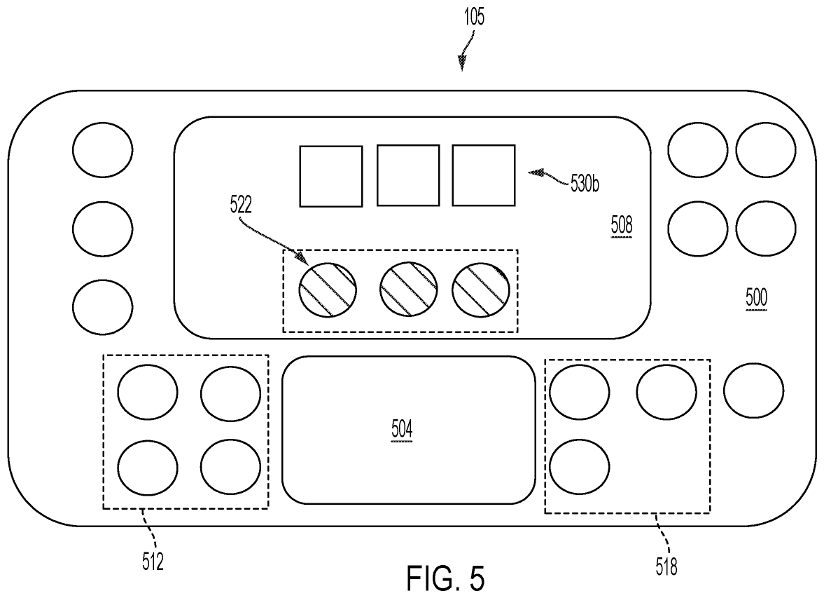

FIG. 5 illustrates another embodiment of a configuration of the input device 105 wherein the graphical user interface 508 includes a set of software buttons 530b outside of input display region 522. In the embodiment shown, set of software buttons 530b have a different shape compared to the software buttons 530 located within the input display region 522. This configuration allows the operator to readily distinguish software buttons within the input display region 522 by shape. Any of software buttons 530b and software buttons 530 may be allocated specific kinds of functions, such as control of touch pad 504. Note that the shape of the hardware buttons 510 included in first area 512 and second area 518, which may also be allocated specific functions related to control of medical observation device 103, have the same shape as the software buttons 530 within input display region 522. In case the hardware buttons 510 included in first area 512 or second area 518 and the software buttons 530 within input display region 522 are not allocated a similar function relating to the control of the medical observation device 103, it would be beneficial to change the shape of the software buttons 530 to easily distinguish the one set of buttons from the other.

Another way to allow the operator to easily distinguish the software buttons 530 within input display region 522 from the other software buttons, such as other software buttons 530b within graphical user interface 508 is to assign a tactile feature to the portion of the graphical user interface 508 where the software buttons 530b and/or software buttons 530 within the input display region 522 is designed to appear. Tactile features can include one or more of a protrusion, a surface having different friction level compared to the other portions of the graphical user interface 508, or other type of irregularity that could be sensed by the operator's tactile sensation.

Yet another way to allow the operator to easily distinguish software buttons within input display region 522, e.g., software buttons 530, from other software buttons within the graphical user interface 508, e.g., software buttons 530b, is to assign haptic feedback features to the software buttons 530 within the input display region 522. Haptic feedback feature can include one or more of a vibration within the input device 105 in response to pushing the software buttons 530 or other type of feedback feature responding to the operator's touching of the software buttons 530. Of course, the haptic feedback can also be provided on other software buttons within the graphical user interface 508, e.g., software buttons 530b, instead of on software buttons within input display region 522, e.g., software buttons 530; or a different haptic feedback can be used to distinguish between the two software buttons.

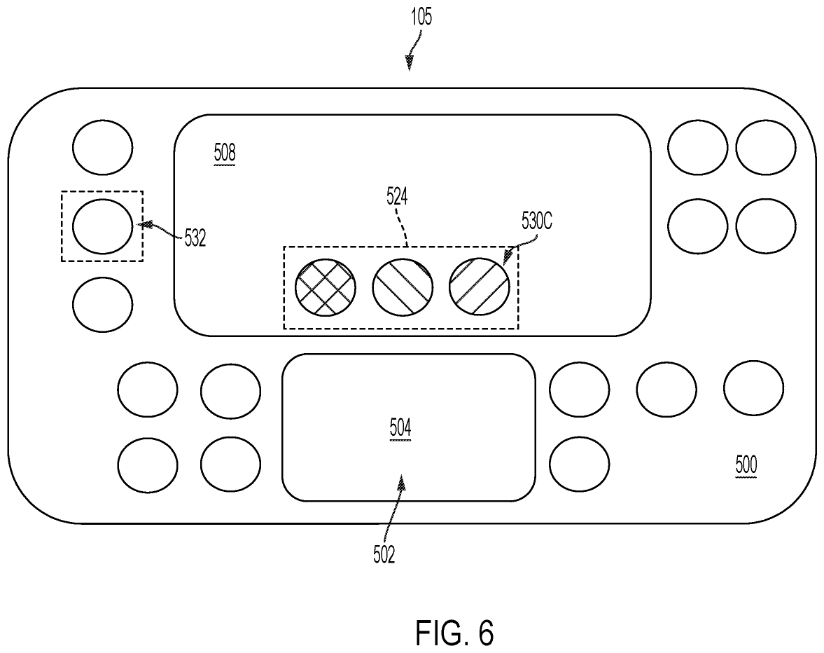

FIG. 6 illustrates another embodiment of a configuration of the input device 105 wherein the graphical user interface within the input display region 524 includes a set of software buttons 530c, which is designed to appear on the graphical user interface 508 in response to an operation of an observation mode setting hardware button 532. An operator may select the preferred observation mode from multiple observation mode setting hardware buttons, or select the preferred observation mode using a single observation mode setting hardware button. In an example embodiment, a single observation mode setting hardware button may interchange the observation modes by the number of times the buttons are pressed.

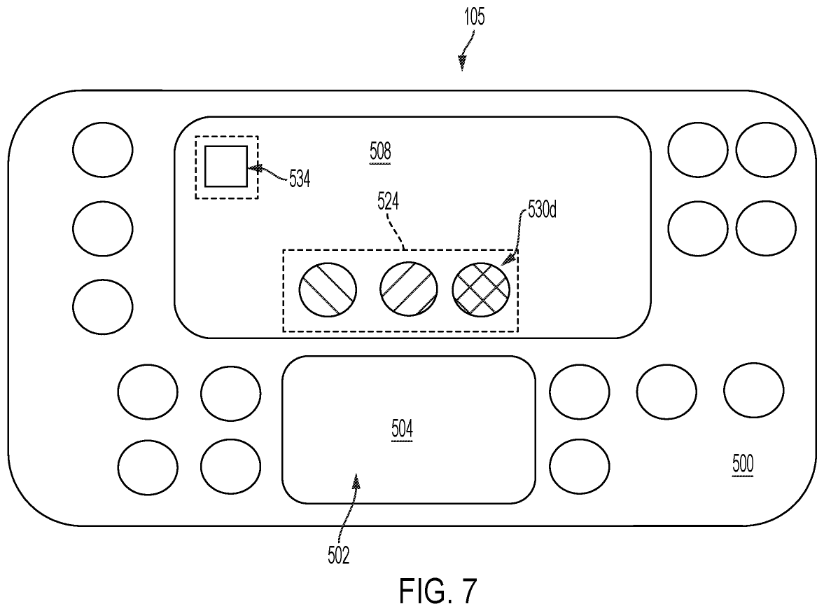

FIG. 7 further illustrates another set of software buttons 530d appearing on the graphical user interface 508, which appears upon the operation of the observation mode setting button 534, which in this case is a software button placed on the graphical user interface 508. As with the FIG. 6 embodiment, the operator may select the preferred observation mode from multiple observation mode setting software buttons, or select the preferred observation mode using a single observation mode setting software button. This configuration allows the operator to easily find the most frequently used buttons in accordance to the observation mode, and place such most frequently used buttons in the vicinity of the pointing device 502, allowing easy and singlehanded operation of input device 105. Also and as indicated in the figures by the different patterns used for the software buttons in the input display region 524, the software buttons may be assigned different control functions and the different control functions can be differentiated by, for example, different identifying patterns, shapes, colors or labeling.

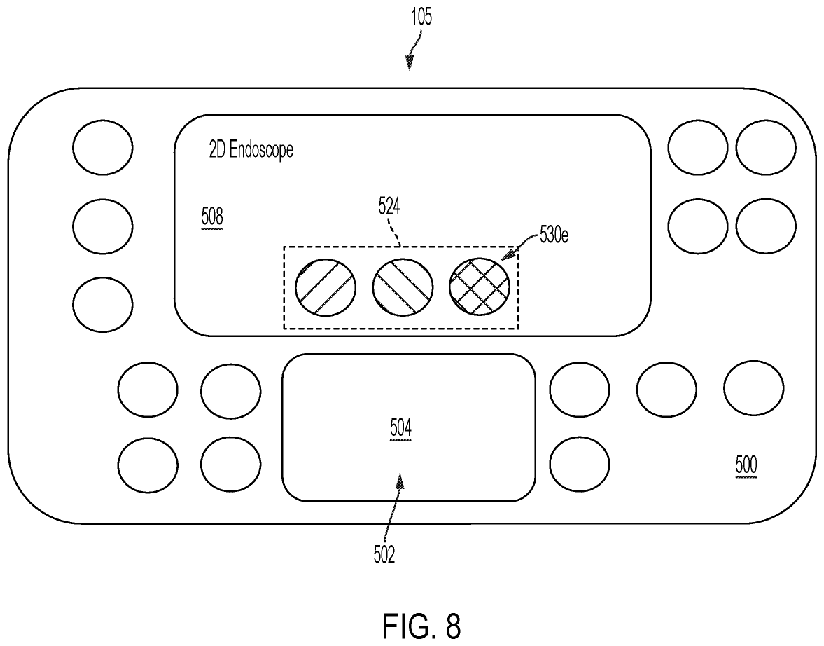

FIG. 8 illustrates another embodiment of a configuration of the input device 105 wherein the graphical user interface within the input display region 524 includes a set of software buttons 530e, which is designed to appear on the graphical user interface 508 (or, optionally, elsewhere on the touch pad 504 or on the interface surface 500) upon detection of the type of medical equipment being connected to the medical observation device, in this case a 2D endoscope.

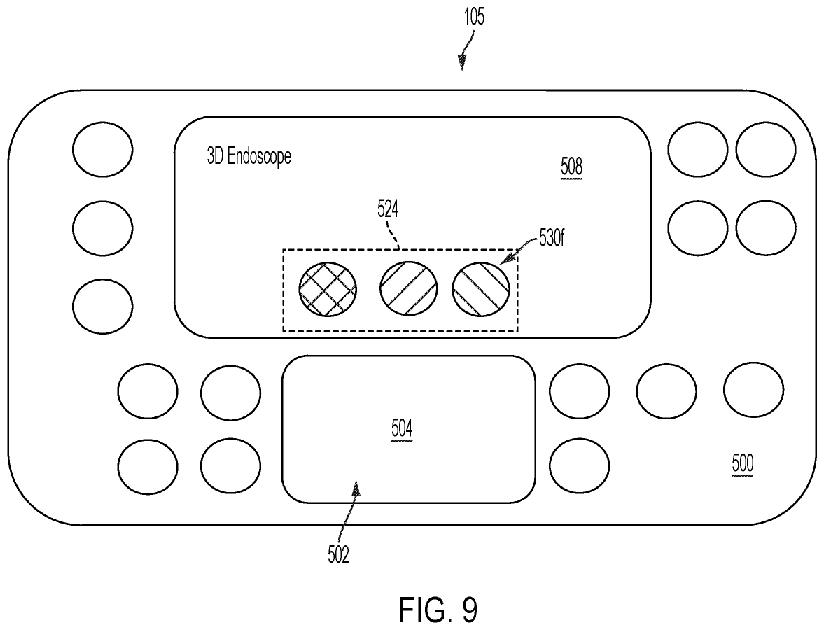

FIG. 9 further illustrates another set of software buttons 530f that appear on graphical user interface 508 upon detection of another type of medical equipment being connected to the medical observation device, in this case a 3D endoscope. Note that the name of the detected medical equipment may also be shown on the graphical user interface 508 (or, optionally, elsewhere on the touch pad 504 or on the interface surface 500). This configuration allows the operator to easily find the most frequently used buttons in accordance to the type of medical equipment the operator is using, and place such most frequently used buttons near the pointing device 502, allowing easy and singlehanded operation of input device 105.

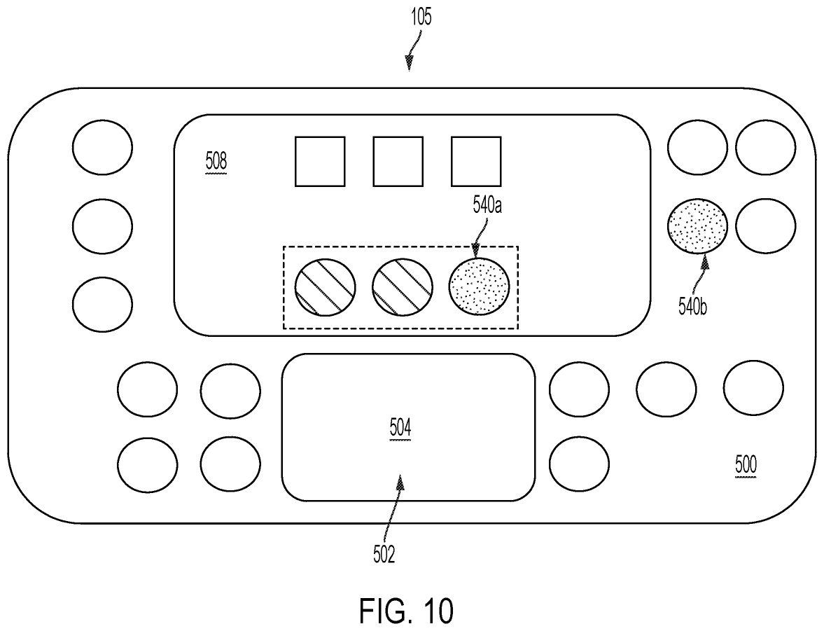

FIG. 10 illustrates inactive buttons designed to appear as software buttons on the graphical user interface 508 (e.g., inactive software button 540a) or as a hardware button on the interface surface 500 (e.g., inactive hardware button 540b). The inactive software buttons 540a appearing on the graphical user interface 508 may be a blacked out button, a greyed out button superimposing the function of the button, or a non-lighted button, all having similar inactiveness to the operator's operation. The inactive hardware buttons 540b are also inactive to the operator's operation, but the inactive status of the inactive hardware buttons 540b may be shown by disabling the light source (i.e. LED) behind the inactive hardware button 540b, in order to distinguish from the other active hardware and software buttons. Enabling inactive buttons in accordance to the observation mode or the type of medical equipment connected to the medical observation device allows less operation error by the operator. Distinguishing inactive buttons from active buttons can improve the operator's usability of the input device 105. In addition to the visual distinguishing aids discussed above, inactivating the haptic feedback function may further improve operator's usability of the input device 105.

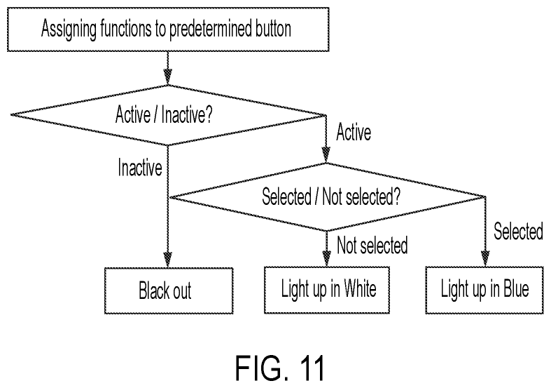

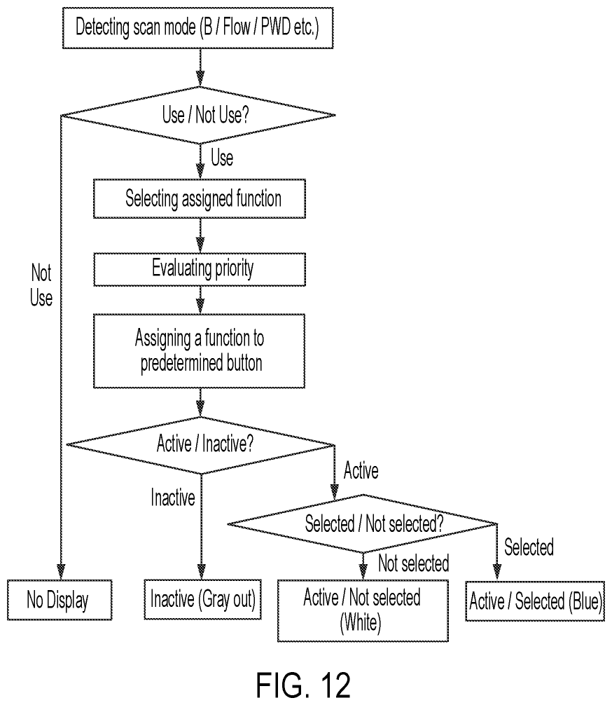

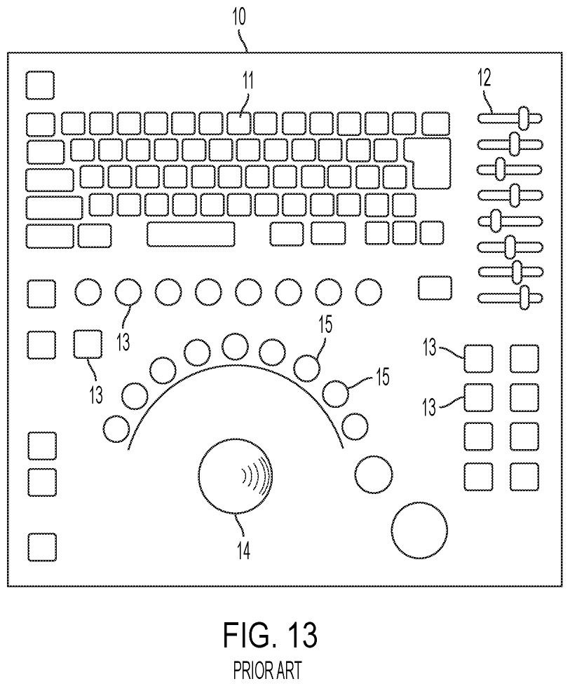

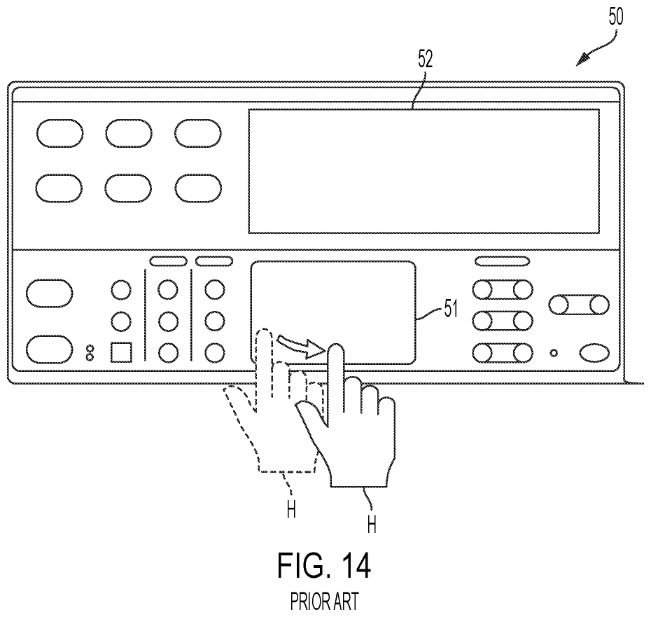

FIG. 11 illustrates a flow chart related to the operation of inactive and active buttons, which may be used for both software and hardware buttons. FIG. 12 illustrates a flow chart related to the operation of inactive and active buttons, more specific to software buttons with a specific assigned function in accordance with the selected observation mode.

Although the present invention has been described in connection with preferred embodiments thereof, it will be appreciated by those skilled in the art that additions, deletions, modifications, and substitutions not specifically described may be made without department from the spirit and scope of the invention as defined in the appended claims.

What is claimed is:

1. An input device for operating a medical observation device, comprising:
an interface surface;
a pointing device arranged on the interface surface, wherein the pointing device is configured to input an operation to the medical observation device;
a first area of the interface surface, wherein the first area is located adjacent a first portion of a periphery of the pointing device and wherein the first area includes at least a first plurality of hardware buttons configured to control functions of the medical observation device;
a second area of the interface surface, wherein the second area is located adjacent a second portion of the periphery of the pointing device and wherein the second area includes at least a second plurality of hardware buttons configured to control functions of the medical observation device; and
a touch panel arranged on the interface surface, wherein the touch panel includes a graphical user interface including an input display region containing at least one software button configured to control a function of the medical observation device, wherein the input display region is located at a first edge portion of the graphical user interface and the first edge portion of the graphical user interface is located adjacent a third portion of the periphery of the pointing device,
wherein, along the periphery of the pointing device, the third portion of the periphery is interposed between the first portion of the periphery and the second portion of the periphery.

2. The input device according to claim 1, wherein, along the periphery of the pointing device, a fourth portion of the periphery is interposed between the first portion of the periphery and the second portion of the periphery.

3. The input device according to claim 2, wherein an area adjacent the fourth portion of the periphery is free of hardware buttons configured to control functions of the medical observation device and is free of software buttons configured to control functions of the pointing device.

4. The input device according to claim 1, wherein the first portion of the periphery and the second portion of the periphery are at opposing sides of the pointing device.

5. The input device according to claim 1, wherein a number of hardware buttons in the first area is different from a number of hardware buttons in the second area.

6. The input device according to claim 1, wherein at least one of the first plurality of hardware buttons is configured to control the operation inputted to the medical observation device by the pointing device.

7. The input device according to claim 6, wherein at least one of the second plurality of hardware buttons is configured to control the operation inputted to the medical observation device by the pointing device.

8. The input device according to claim 7, wherein the operation of the pointing device inputted by the at least one hardware button is used in a plurality of modes of the medical observation device.

9. The input device according to claim 6, wherein the operation of the pointing device inputted by the at least one hardware button is used in a plurality of modes of the medical observation device.

10. The input device according to claim 1, wherein at least one of the second plurality of hardware buttons is configured to control the operation inputted to the medical observation device by the pointing device.

11. The input device according to claim 1, wherein the operation of the pointing device inputted by the hardware buttons is selected from the group consisting of a pointer function, a measurement function, a set function, a comment function, and a delete function.

12. The input device according to claim 1, wherein the function of the pointing device associated with at least one software button changes in accordance with a mode of the medical observation device.

13. The input device according to claim 12, wherein the mode of the medical observation device is selected using a mode button arranged in a mode display region of the graphical user interface or adjacent to the touch panel.

14. The input device according to claim 1, wherein the function of the pointing device associated with the at least one software button is selected from the group consisting of scroll, rotation, focus change depth, ROI position change, and ROI size change.

15. The input device according to claim 1, wherein the function of the pointing device associated with the at least one software button changes based on a type of a medical device connected to the medical observation device.

16. The input device according to claim 1, wherein the input display region of the graphical user interface is located at a twelve o'clock position relative to the periphery of the pointing device.

17. The input device according to claim 16, wherein the first area is located at a nine o'clock position relative to the periphery of the pointing device and the second area is located at a three o'clock position relative to the periphery of the pointing device.

18. The input device according to claim 1, wherein the interface surface associated with one or more of (i) the pointing device, (ii) at least one of the first plurality of hardware buttons, (iii) at least one of the second plurality of hardware buttons, and (iv) the at least one software button includes a tactile feature.

19. The input device according to claim 1, wherein the interface surface associated with one or more of (i) the pointing device, (ii) at least one of the first plurality of hardware buttons, (iii) at least one of the second plurality of hardware buttons, and (iv) the at least one software button includes a haptic feedback feature.

20. The input device according to claim 1, wherein the pointing device is one of a touch pad, a track ball, a pointing stick, or a joystick.

21. The input device according to claim 1, wherein the hardware and the software buttons controlling the function of the pointing device have the same shape.

22. The input device according to claim 1, wherein the first area of the interface surface, the second area of the interface surface, and the input display region of the graphical user interface are all at a position on the interface surface that is within 15 cm from a center of the pointing device.

23. The input device according to claim 1, wherein the first area of the interface surface, the second area of the interface surface, and the input display region of the graphical user interface are all co-located within an operating area of the interface surface operable by a hand of an operator without changing a location of the hand relative to the interface surface.

24. The input device according to claim 1, wherein the first area of the interface surface, the second area of the interface surface, and the input display region of the graphical user interface are all co-located within an operating area of the interface surface of less than 100 cm².

25. The input device according to claim 24, wherein the operating area of the interface surface is less than 90 cm².

26. The input device according to claim 24, wherein a shape of the operating area of the interface surface is selected from one of a quadrilateral, a circle and an oval.

27. A medical observation system, comprising:
a medical device;
a medical observation device connected to the medical device; and
the input device according to claim 1 connected to the medical observation device.

28. The medical observation system according to claim 27, wherein the medical device includes an endoscope.

29. A method of operating the medical observation system according to claim 27, the method comprising:
inputting an operation to the medical observation device via the pointing device arranged on the interface surface of the input device;
controlling functions of the medical observation device via the first plurality of hardware buttons arranged in the first area of the interface surface located adjacent the first periphery of the pointing device;
controlling functions of the medical observation device via the second plurality of hardware buttons arranged in the second area of the interface surface located adjacent the second periphery of the pointing device; and
controlling functions of the medical observation device via the at least one software button contained in the input display region of the graphical user interface included in the touch panel arranged on the interface surface of the input device, where the input display region is located at the first edge portion of the graphical user interface and the first edge portion of the graphical user interface is located adjacent the third portion of the periphery of the pointing device,
wherein the third portion of the periphery is interposed between the first portion of the periphery and the second portion of the periphery of the pointing device.
